(19) United States
(12) Patent Application Publication
(10) Pub. No.: US 2021/0379026 A1
(43) Pub. Date: Dec. 9, 2021

(54) METHODS FOR TREATING OR PREVENTING SKIN CONDITIONS

(71) Applicant: Drexel University, Philadelphia, PA (US)

(72) Inventors: Christina L. Chung, Philadelphia, PA (US); Christian Sell, Conshohocken, PA (US); Ibiyonu O. Lawrence, Whitehouse Station, NJ (US)

(21) Appl. No.: 17/283,754

(22) PCT Filed: Oct. 10, 2019
(86) PCT No.: PCT/US2019/055492

§ 371 Date: Apr. 8, 2021

Related U.S. Application Data
(60) Provisional application No. 62/744,486, filed on Oct. 11, 2018.

(51) Int. Cl. A61K 31/436; A61P 17/00; A61K 9/00; A61K 9/06

(52) U.S. Cl. CPC A61K 31/436; A61K 9/06; A61K 9/0014; A61P 17/00

(57) ABSTRACT

The present invention includes methods and compositions for preventing, ameliorating, and treating certain skin disorders caused due to UV-induced, age-related, and post-inflammatory cutaneous changes. The skin disorders include, in non-limiting examples, static wrinkles, fine wrinkles, loss of skin tone, ephelides, melasma, senile purpura, UV-damage to the skin, static rhytides, prominent hand veins, field cancerization of epithelium such as in squamous cell carcinoma in situ, keloids, and imbalance in ratio of collagen isoforms. In certain embodiments, the compositions useful within the invention comprise a therapeutically effective amount of a rapamycin and a dermatologically acceptable carrier.

### CROSS-REFERENCE TO RELATED APPLICATIONS

This application claims priority under 35 U.S.C. § 119(e) to U.S. Provisional Patent Application No. 62/744,486, filed Oct. 11, 2018, which application is incorporated herein by reference in its entirety.

### BACKGROUND OF THE INVENTION

Several skin disorders are caused due to cutaneous changes triggered by environmental factors, hormonal changes, aging, and inflammatory episodes. Some of these disorders include, for example, ephelides, solar lentigos, melasma, senile purpura, fine lines, static rhytides, prominent hand veins, actinic keratoses and field cancerization of the epithelium, formation of keloids, and imbalance in the ratio of collagen isoforms.

Ephelides, solar lentigos, melasma, actinic keratoses and squamous cell carcinoma in situ are ailments resulting from sun exposure. Ephelides, commonly known as freckles, are small pigmented lesions. Solar lentigos are larger hyperpigmented lesions and melasma is even larger, patchy brown, tan, or blue-gray facial skin discoloration. Field cancerization refers to large areas of sun-exposed skin that display actinic damage and epidermal dysplasia. Actinic keratoses, generally appear as pink, scaly papules or plaques and often arise in areas of field cancerization. Left untreated, field cancerization and actinic keratoses may progress to squamous cell carcinoma.

Senile purpura, fine lines, static rhytides, and prominent hand veins are the conditions that generally affect aging skin. Senile purpura usually occurs following a minor trauma and is a common, benign condition characterized by recurrent formation of purple ecchymosis (bruises) caused by bleeding into the dermis by fragile capillaries that are susceptible to insult because of thinning of the dermis, or scaffolding, of the skin. Static rhytides are wrinkles that remain unchanged with muscular movements and prominent hand veins is a condition where veins become prominent either due to very low body fat or because of the thinning of the structures that support the hand veins. Fine lines are a result of an irregular dermis and a decrease in the ability to retain water in the epidermis caused by sun damage and other external factors.

Keloids are scars composed mainly of either type III (early) or type I (late) collagen. Keloids form from overgrowth of granulation tissue (collagen type 3) at the site of a healed skin injury, which is then slowly replaced by collagen type 1. Keloids are an aberrant scar reaction that results in firm, hyperpigmented plaques, whose borders extend beyond the area of primary injury. Keloids disproportionately affect nonwhite persons and are often pruritic, tender, and unsightly. Additionally, keloids can cause tightness, as well as limit the range of motions, if they occur near a joint, such as, for example, a knee or an ankle.

There is a need in the art for compositions and methods that can be used to treat or prevent certain dermal disorders caused by UV-induced, age-related, and post-inflammatory cutaneous changes. The present invention addresses this need.

### BRIEF SUMMARY OF THE INVENTION

In one embodiment, the invention provides a method of treating and/or preventing a dermal disorder in a mammalian subject in need thereof. The method comprises topically administering to the subject a composition comprising a therapeutically effective amount of rapamycin, or a salt, solvate, enantiomer or diastereoisomer thereof. In certain embodiments, the dermal disorder is at least one selected form the group consisting of static wrinkles, fine wrinkles, loss of skin tone, ephelides, melasma, senile purpura, static rhytides, prominent hand veins, field cancerization of epithelium such as in squamous cell carcinoma, keloids, and imbalance in ratio of collagen isoforms.

In another embodiment, the composition comprises about 1% to about 0.0001% by weight of the rapamycin, or a salt, solvate, enantiomer or a diastereoisomer thereof.

### BRIEF DESCRIPTION OF THE DRAWINGS

For the purpose of illustrating the invention, certain embodiments of the invention are depicted in the drawings. However, the invention is not limited to the precise arrangements and instrumentalities of the embodiments depicted in the drawings.

FIG. 1 shows that rapamycin treatment reduces p16 levels in human skin.

FIG. 2 shows that the rapamycin-treated biopsy exhibits reduction in the histological markers of age/damaged skin compared to the placebo-treated biopsy.

FIG. 3 shows that the rapamycin-treated biopsy exhibits more orderly arrangement of the epithelial layer compared to the placebo treated biopsy.

FIG. 4 shows that the rapamycin-treated biopsy exhibits a more orderly arrangement of collagen fibers and a fewer solar elastoses compared to the placebo-treated biopsy.

FIG. 5 shows that the rapamycin-treated skin is more focally located in the basal layer of the skin, while the placebo-treated skin shows cytokeratin 5/6 staining in the stratum granulosum, indicative of incomplete differentiation which is typical of an aged skin.

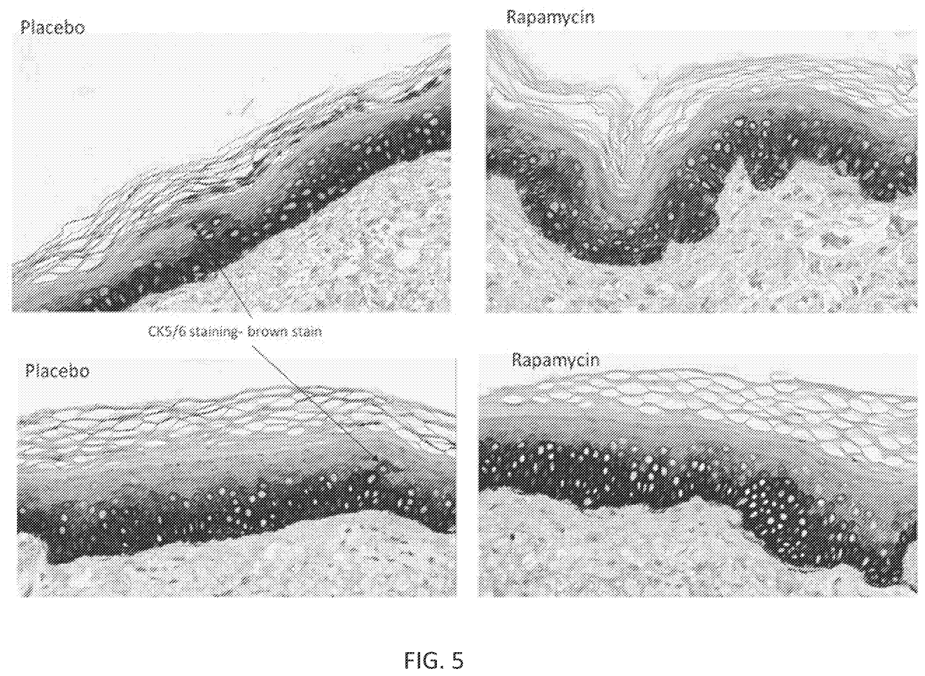

FIG. 6 shows cytokeratin 5/6 (CK 5/6) staining demonstrating a positive staining in the stratum granulosum and extending into the stratum corneum in the placebo-treated skin, while the pattern of CK 5/6 staining is more restricted to the basal layer in the rapamycin-treated biopsy.

FIG. 7 shows clinical improvement in a 67 years old woman following the application of rapamycin cream for 6 months. The placebo-treated left hand is also shown for comparison.

FIG. 8 shows significant reduction in purpura following 4.5 months of rapamycin treatment.

FIGS. 9A-9C show that rapamycin protects against UV-induced cell death.

### DETAILED DESCRIPTION OF THE INVENTION

The present invention relates, in one aspect, to a method of preventing and/or treating various dermal and epidermal disorders including, but not limited to, ephelides, melasma, senile pupura, static rhytides, prominent hand veins, field cancerization of the epithelium, keloid formation and imbalance in the ratio of collagen isoforms.

In certain embodiments, the invention provides a composition comprising a therapeutically effective amount of rapamycin, or a salt, solvate, enantiomer or a diastereoisomer thereof. In other embodiments, the composition comprises rapamycin, or a salt, solvate, enantiomer or diastereoisomer thereof, as the only ingredient that is active against the dermal conditions contemplated herein. In yet other embodiments, the composition comprises rapamycin, or a salt, solvate, enantiomer or diastereoisomer thereof, as the only ingredient that is present in a sufficient concentration and/or amount to be active against the dermal conditions contemplated herein. In yet other embodiments, the composition is formulated for topical administration.

Proper differentiation is critical to skin functions such as providing a protective layer preventing water loss, protection from UV damage due to sun exposure, and serving a barrier function against pathogens. These protective functions require an orderly differentiation of cells within the epidermis. With aging, the differentiation pattern in the skin is disrupted creating a dysfunctional barrier, thereby leading to a fragile skin that is prone to wounds, tears, and/or UV-damages. As demonstrated herein, the nanomolar concentrations of rapamycin produce a more orderly differentiation within the epidermis of human skin along with a reduction in the markers of aging and cellular dysfunction. A key protein involved with the aging of cells in dermis and epidermis, p16^{INK4A}, is reduced in expression. This protein is associated with aging related changes in the skin (Waaijer, et al., 2016, J Gerontol A Biol Sci Med Sci. 71(8):1022-1028; Waaijer, et al., 2012, Aging Cell 11(4):722-725). In addition, histologic changes associated with UV-damage in the skin are also reduced by rapamycin treatment. These changes translate into clinical improvements in skin-tone and wrinkling in older individuals.

### Definitions

As used herein, each of the following terms have the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics and chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, ±5%, ±1%, or ±0.1% from the specified value, as such variations are appropriate to perform the disclosed methods.

As used herein, “dermatologically acceptable carrier” or “dermatologically acceptable excipient” refers to the compositions or components that are suitable for use in contact with human keratinous tissue without undue toxicity, incompatibility, instability, allergic response, and the like.

A “disease” is a state of health of an animal wherein the animal cannot maintain homeostasis, and wherein if the disease is not ameliorated, the animal's health continues to deteriorate. A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a compound are used interchangeably to refer to the amount of the compound sufficient to provide a beneficial effect to the subject to which the compound is administered. The term to “treat,” as used herein, means reducing the frequency with which symptoms are experienced by a patient or subject or administering an agent or compound to reduce the severity with which symptoms are experienced. An appropriate therapeutic amount in any individual case may be determined by one of ordinary skill in the art using routine experimentation.

As used herein, an “instructional material” includes a publication, a recording, a diagram, or any other medium of expression that can be used to communicate the usefulness of a compound, composition, assay or method of the invention in a kit for suppressing or reducing systemic immune response in a subject. The instructional material of the kit of the invention can, for example, be affixed to a container which contains the identified compound, composition, assay, or methods of the invention or be shipped together with a container that contains the identified compound, composition, assay, or method. Alternatively, the instructional material can be shipped separately from the container with the intention that the instructional material and the compound, composition, assay, or method be used cooperatively by the recipient.

As used herein, the term “modulate” means, with respect to disease states or conditions associated with binding of a compound of the present invention to a receptor contemplated in the present invention, to produce, either directly or indirectly, an improvement or lessening of a condition or disease state which was, prior to administration of a compound according to the present invention, sub-optimal and in many cases, debilitating and even life threatening. Modulation may occur by virtue of agonist activity, antagonist activity or mixed agonist/antagonist activity (depending on the receptor site).

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutical composition” or “composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary, intracranial and topical administration. In certain embodiments, the administration comprises topical administration.

As used herein, a “subject” refers to a human or non-human mammal. Non-human mammals include, for example, livestock and pets, such as ovine, bovine, porcine, canine, feline and murine mammals. In certain embodiments, the subject is human.

As used herein, “topical administration” or “topical application” refers to a medication applied to body surfaces such as the skin or mucous membranes.

As used herein, the term “treatment” or “treating” is defined as the application or administration of a therapeutic agent, i.e., a composition useful within the invention (alone or in combination with another pharmaceutical agent), to a subject, or application or administration of a therapeutic agent to an isolated tissue or cell line from a subject (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder or the potential to develop the disease or disorder. Such treatments may be specifically tailored or modified, based on knowledge obtained from the field of pharmacogenomics.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

In one embodiment, the composition of the invention comprises a therapeutically effective amount of rapamycin, or salt, solvate, enantiomer or diastereoisomer thereof Rapamycin is also known as (3S,6R,7E,9R,10R,12R,14S,15E,17E,19E,21S,23S, 26R,27R,34aS)-9,10,12,13,14,21,22,23,24,25,26,27,32,33,34,34a-Hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4-oxaazacyclo hentriacontine-1,5,11,28,29(4H,6H,31H)-pentone, and has the following structure:

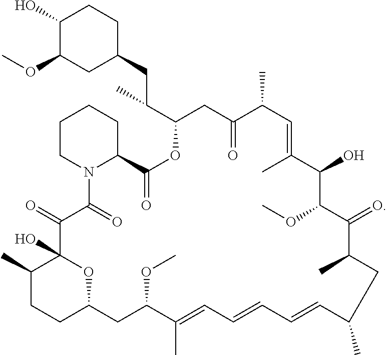 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In certain embodiments, modified form of rapamycin with improved delivery to specific intracellular compartments or organelles, such as the mitochondria, the nucleus, the lysosome, and/or the endoplasmic reticulum can be used. Modified forms of rapamycin include, ester, amide, ether derivatives, and the like.

Other mTORC1 inhibitors aside from rapamycin are also useful within the methods presented herein to achieve the therapeutic effects described herein. In certain embodiments, the mTORC1 inhibitor is at least one selected from the group consisting of BEZ235, everolimus, AZD8055, Temsirolimus, KU-0063794, PI-103, Torkinib, Tacrolimus, Ridaforolimus, INK-128, Voxtalisib, Torin-1, Omipalisib, OSI-027, PF-04691502, Apitolisib, GSK1059615, WYE-354, Gedatolisib, AZD-2014, Torin-2, WYE-125132, BGT226, Palomid-529, PP121, WYE-687, CH5132799, Way-600, ETP-46464, GDC-0349, XL388, and Zotarolimus, or a salt, solvate, enantiomer or diastereoisomer thereof. In other embodiments, the mTORC1 inhibitor is at least one selected from the group consisting of rapamycin, Ridaforolimus, and Everolimus, or a salt, solvate, enantiomer or diastereoisomer thereof. In yet other embodiments, the mTORC1 inhibitor is rapamycin, or a salt, solvate, enantiomer or diastereoisomer thereof. Therapeutically effective amounts of any of the mTORC1 inhibitors described herein can be from about 0.0001% to about 1%, 0.0005% to about 0.95%, about 0.001% to about 0.85%, 0.002% to about 0.75%, about 0.005% to about 0.5%, about 0.008% to about 0.25%, about 0.01% to about 0.2%, about 0.02% to about 0.15%, about 0.0001% to about 0.001%, about 0.0001% to about 0.01%, or about 0.03% to about 0.1% (w/w) of the composition. The therapeutically effective amount of an mTORC1 inhibitor that can be used in the compositions described herein can be about 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or about 0.0001% (w/w) of the composition.

In certain embodiments, the therapeutically effective amount of the rapamycin in the composition ranges from about 0.0001% to about 1%, 0.0005% to about 0.95%, about 0.001% to about 0.85%, 0.002% to about 0.75%, about 0.005% to about 0.5%, about 0.008% to about 0.25%, about 0.01% to about 0.2%, about 0.02% to about 0.15%, about 0.0001% to about 0.001%, about 0.0001% to about 0.01%, or about 0.03% to about 0.1% (w/w) of the composition. In certain embodiments, the therapeutically effective amount of rapamycin in the composition ranges from about 0.0001% to about 0.001% (w/w). In other embodiments, the therapeutically effective amount by weight of the rapamycin in the composition ranges from about 0.0001% to about 0.01% (w/w).

In certain embodiments, the therapeutically effective amount of rapamycin in the composition is about 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or about 0.0001% (w/w). Compositions containing rapamycin at or below 0.001% w/w, in certain embodiments, do not inhibit dermal cellular growth, and the proliferative potential of dermal cells is maintained. Compositions containing rapamycin at or below 0.001% w/w, in certain embodiments, prevent or reduce senescence in dermal cells. In certain embodiments, compositions containing rapamycin in an amount of about 0.001%, 0.0009%, 0.0008%, 0.0007%, 0.0006%, 0.0005%, 0.0004%, 0.0003%, 0.0002%, or about 0.0001% (w/w) do not inhibit dermal cell growth, maintain the proliferative potential of dermal cells, and prevent or reduce senescence in dermal cells.

In certain embodiments, the composition of the invention further comprises a dermatologically acceptable carrier. The compositions of the present invention may comprise from about 60% to about 99.9%, alternatively from about 70% to about 95%, and alternatively from about 80% to about 90%, of a dermatologically acceptable carrier. In certain embodiments, the dermatologically acceptable carrier is at least selected from the group consisting of solvent, lubricant, emollient, emulsifier, moisturizer, thickening wax, softener, fragrance, preservative, and artificial color(s). In other embodiments, the dermatologically acceptable carrier is at least one selected from the group consisting of water, fatty alcohols, and volatile organic alcohols. One non-limiting example of the dermatologically acceptable carrier is petrolatum.

### Methods

In one embodiment, the invention is a method for prevention and/or early treatment of UV-induced dyspigmentation caused by ephelides and melasma. In another embodiment, the invention provides a method for prevention and/or treatment of age-related dermal disorders such as, but not limited to, senile purpura, static rhytides, and/or prominent hand veins. In yet another embodiment, the invention provides a method for prevention and/or treatment of field cancerization of epithelium such as in squamous cell carcinoma in situ. In yet another embodiment, the invention provides a method for prevention and/or treatment of keloids. In yet another embodiment, the invention further provides a method for maintaining optimum ratio of collagen isoforms so as to prevent aging. In yet another embodiment, the invention further provides a kit for treating or preventing a dermal disorder caused due to cutaneous changes occurring in a mammalian subject in need thereof.

In certain embodiments, the methods comprises topically administering to the subject a therapeutically effective amount of rapamycin, which is optionally formulated in a dermally acceptable composition.

In certain embodiments, the composition comprises a therapeutically effective amount of rapamycin. In yet other embodiments, the composition further comprises a dermatologically acceptable carrier. In yet other embodiments, the composition is applied topically to the affected skin area of the subject.

In certain embodiments, topical formulations of the compositions contemplated herein are used for treating UV-induced, age-related as well as post inflammatory cutaneous changes.

In certain embodiments, the invention provides a topical cream comprising a therapeutically effective amount of rapamycin for treating or preventing UV-induced, age-related as well as post inflammatory cutaneous changes.

In certain embodiments, the cutaneous changes are evaluated by measurement of senescence related proteins such as p16^{INK4A}, markers of differentiation such as cytokeratin 5/6, organization of collagen fibers, clinical measures such as severity of wrinkling and skin-tone.

### Formulations

The relative amounts of the active ingredient, the dermatologically acceptable carrier, and any additional ingredients in a pharmaceutical composition of the invention will vary, depending upon the identity, size, and condition of the subject treated. By way of example, the composition may comprise between about 0.0001% and about 1% (w/w) of rapamycin. In certain embodiments, the composition comprises about 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or about 0.0001% (w/w) of rapamycin.

In certain embodiments, the therapeutically effective amount by weight of the rapamycin in the composition ranges from about 0.0001% to about 1%, 0.0005% to about 0 95%, about 0.001% to about 0.85%, 0.002% to about 0.75%, about 0.005% to about 0.5%, about 0.008% to about 0.25%, about 0.01% to about 0.2%, about 0.02% to about 0.15%, or about 0.03% to about 0.1%.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

The composition of the invention can be administered to a mammal as frequently as several times daily, or it may be administered less frequently, such as once a day, once a week, once every two weeks, once a month, or even less frequently, such as once every several months or even once a year or less.

Dosing regimens for administering the compositions of the invention may be once a day or twice a day. The frequency of the application and the concentration of the active agent is dependent on the skin condition and the response of the dermis. Application can be continued to achieve the desired effect on the dermis and the frequency of application can be reduced after a satisfactory result has been obtained. In certain embodiments, the administration requires a minimum of 6-8 weeks to achieve results. Applications can continue beyond the initial 6-8 week period to obtain continued improvement and the frequency of application can be reduced once this result has been achieved. Applications may continue over the course of years with variable levels of application based upon the relative severity of lesions at any one time.

It is understood that the amount of the composition of the invention dosed per day may be administered, in non-limiting examples, every day, every other day, every 2 days, every 3 days, every 4 days, or every 5 days. The frequency of the dose will be readily apparent to the skilled artisan and will depend upon any number of factors, such as, but not limited to, the type and severity of the disease being treated, the type and age of the animal, and so forth.

In certain embodiments, the compositions of the invention are formulated using one or more dermatologically acceptable excipients or carriers. In certain embodiments, the pharmaceutical compositions of the invention comprise a therapeutically effective amount of a rapamycin and a dermatologically acceptable carrier. Dermatologically acceptable carriers, which are useful, include, but are not limited to, glycerol, water, saline, ethanol and other dermatologically acceptable salt solutions such as phosphates and salts of organic acids. Examples of these and other dermatologically acceptable carriers are described in Remington's Pharmaceutical Sciences (1991, Mack Publication Co., New Jersey).

The compositions of the present invention may comprise from about 0.01% to about 1%, alternatively from about 1% to about 10%, and alternatively from about 10% to about 50%, of a dermatologically acceptable carrier. In certain embodiments, the dermatologically acceptable carrier is at least selected from the group consisting of solvent, lubricant, emollient, emulsifier, moisturizer, thickening wax, softener, fragrance, preservative, and artificial color(s). In other embodiments, the dermatologically acceptable carrier is at least one selected from the group consisting of water, fatty alcohols, and volatile organic alcohols. One non-limiting example of the dermatologically acceptable carrier is petrolatum.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for topical administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and/or a chelating agent such as EDTA. Preferred antioxidants for some compounds are BHT, BHA, α-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include aminopolycarboxylic acid salts (e.g. disodium ethylenediaminetetraacetic acid) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Such formulations may be applied to the skin directly or through the use of swabs, applicators, spatulas and the like, as well as in the form of transdermal patches. In certain embodiments, the patch minimizes loss of pharmaceuticals through washing, friction, scratching and/or rubbing of the skin. In other embodiments, the patch increases absorption of the pharmaceutical through the skin, while minimizing the exposure of the skin to the pharmaceutical.

Topically administrable formulations contemplated within the invention may, for example, comprise from about 0.0001% to about 1% (w/w) a rapamycin, although the concentration of the rapamycin may be as high as its solubility limit in the solvent. In one specific embodiment, the composition comprises from about 0.0001% to about 0.1% (w/w) a rapamycin. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, U.S. Pat. No. 6,323,219).

In alternative embodiments, the topical formulation further comprises other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the formulation and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the topical formulation may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

Additional non-active ingredients in the topical formulation are well known in the art. These ingredients include, but are not limited to, humectants, emollients, pH stabilizing agents, chelating agents, gelling agents, thickening agents, emulsifiers, binders, buffers, carriers, anti-oxidants, etc. Additional examples of such ingredients are included in the U.S. Food & Drug Administration, Inactive Ingredients for Approved Drugs, available online. Addition discussion and potential non-active ingredients that may be included in formulations can be found in “The Science and Practice of Pharmacy”, 21st Edition, Lippincott Williams & Wilkins, Philadelphia, Pa. (2006).

In certain embodiments, a gel formulation of the invention comprises inorganic substances, such as aluminum salts or organic polymers of natural or synthetic origin to allow release of rapamycin.

In yet other embodiments, a solution or spray formulation of the invention comprises a polymeric solution of drug which is sprayed over the intact skin, providing a sustained release of rapamycin from the polymeric matrix.

In yet other embodiments, a cream or lotion formulation of the invention comprises polyoxyethylene, glycerol, paraffin, propylene glycol, and glycerol.

In yet other embodiments, an ointment formulation of the invention comprises petroleum, paraffin, steryl alcohol, and cholesterol.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydroxypropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for topical administration, such as liniments, lotions, oil-in-water or water-in-oil emulsions such as creams, ointments or pastes, transdermal patches, and solutions or suspensions that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material which provides sustained release properties to the compounds.

In certain embodiments of the invention, the compositions of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 24 hours, about 48 hours, about 72 hours and any or all whole or partial increments thereof after drug administration after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following examples. These examples are provided for the purpose of illustration only, and the invention is not limited to these examples, but rather encompasses all variations that are evident as a result of the teachings provided herein.

### Materials and Methods

Unless otherwise noted, all cell lines, starting materials, reagents, and cell lines were obtained from commercial suppliers and used without further manipulation.

Patients were provided with a proprietary topical formulation of rapamycin (0.001%; equivalent to 10 μM) or a placebo formulation that is identical with the exception that rapamycin was not included. For seborrheic keratosis, patients were instructed to apply the formulation to a localized area of the skin up to 1 cm around the lesion, in the evening before bed. Patients were instructed to apply the cream to the dorsal side of the hand once a day, in the evening before bed.

The cream included the following ingredients: Polyoxyethylene (40) stearate, Polyoxyethylene (20) sorbitan monooleate, glycerol monostearate, paraffin, cetyl alcohol, mineral oil, water, propylene glycol, sorbitol, glycerol, methylparaben.

Patients were provided with a 4 week supply of creams labeled by a number with no indication of whether they contained rapamycin. Patients were instructed to contact the study personnel if any irritation or negative reaction occurred at any time.

### Example 1

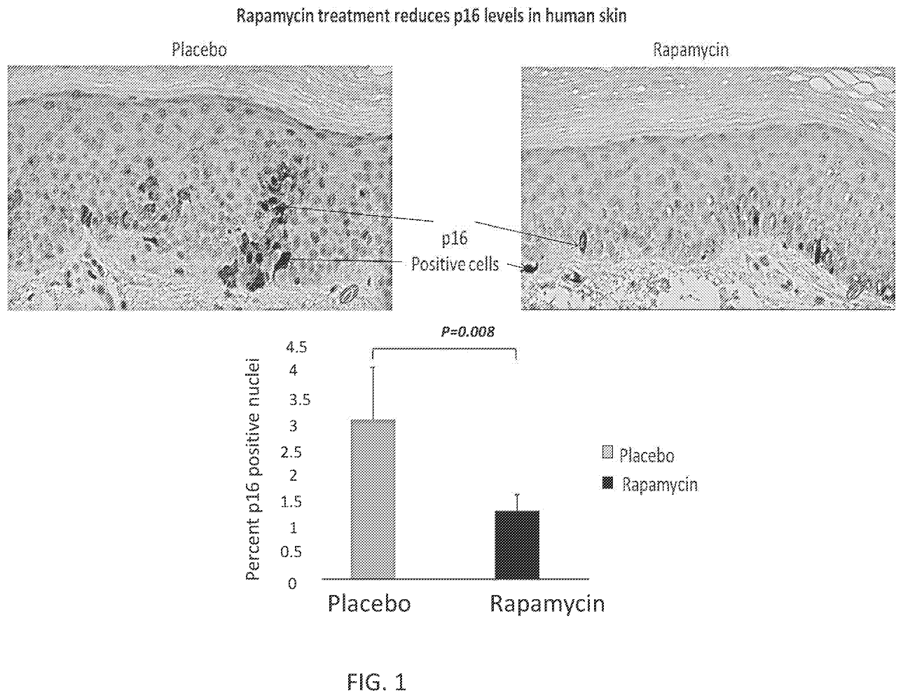

Human skin was treated with a formulation of 1 micromolar rapamycin or an identical formulation containing a vehicle control for 240 days. 0.5 cc of the formulation was applied daily. Skin biopsies (N=8) were processed for immunohistochemistry and nuclear p16 was quantified using the Leica Aperio software system. As seen from FIG. 1, the rapamycin treatment reduced the p16 levels in human skin.

### Example 2

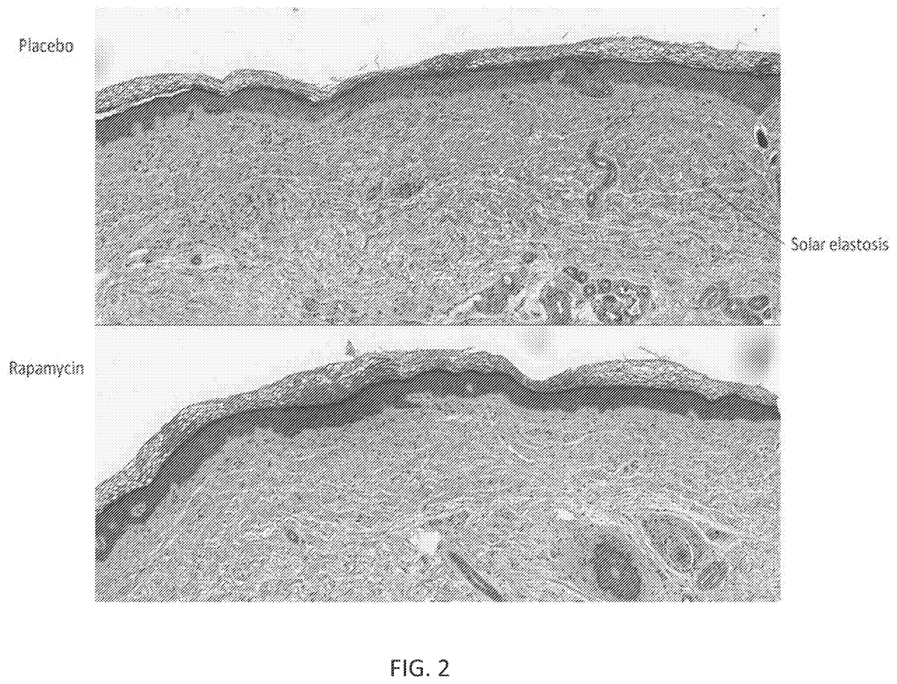

Hematoxylin Eosin stain of human skin biopsies from the dorsal hand of a patient following the application of a topical cream either containing rapamycin (10 μM) or placebo are shown in FIG. 2. Histologic evidence of actinic (solar) elastoses is indicated with an arrow. Similar to that shown in FIG. 2, a reduction in the presence of these histological markers of aged/damaged skin was noted in multiple patient biopsies.

### Example 3

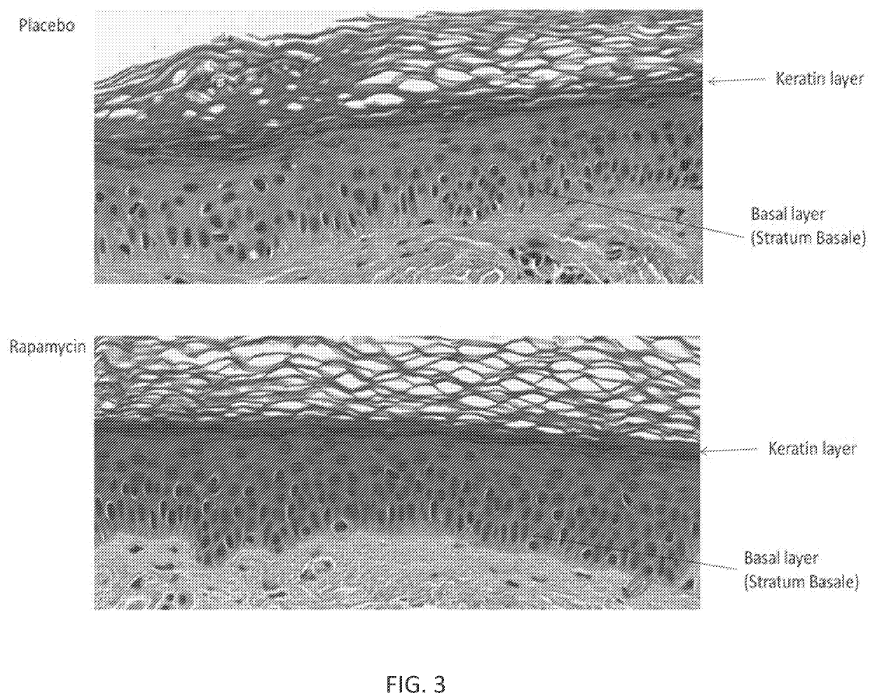

40× magnified images of human skin biopsies from the dorsal hand of a patient following the application of a topical cream either containing rapamycin (10 μM) or placebo are shown in FIG. 3. Rapamycin treated biopsies show a more orderly arrangement of the epithelial layer, including apical placement of nuclei within the basal layer and a more prominent keratin layer.

### Example 4

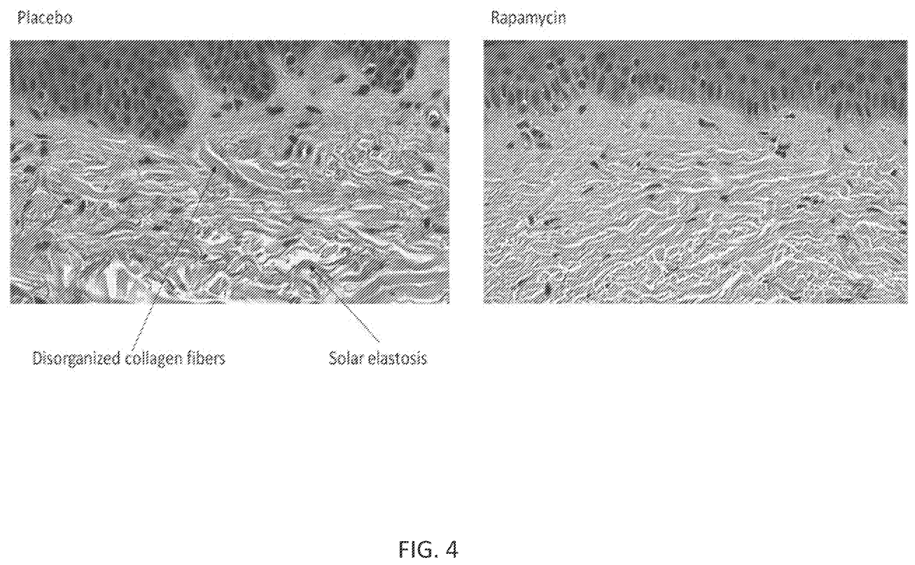

40× magnified images of human skin biopsies from the dorsal hand of a patient following the application of a topical cream either containing rapamycin (10 μM) or placebo are shown in FIG. 4. Untreated skins shows signs of UV damage in the form of purple staining elastin fibers known as solar elastoses and disorganized collagen fibers. Rapamycin treated biopsies show a more orderly arrangement of collagen fibers and fewer solar elastoses.

### Example 5

Human skin biopsies from the dorsal hand of a patient following the application of a topical cream either containing rapamycin (10 μM) or placebo were stained with antibodies recognizing cytokeratin 5/6 (brown staining), which is a marker for basal cells in the epidermis. Note staining in rapamycin treated skin is more focally located in the basal layer of the skin, while the skin receiving placebo treatment shows cytokeratin 5/6 (CK 5/6) staining in the stratum ganulosum, which is indicative of incomplete differentiation that is typical of an aged skin.

### Example 6

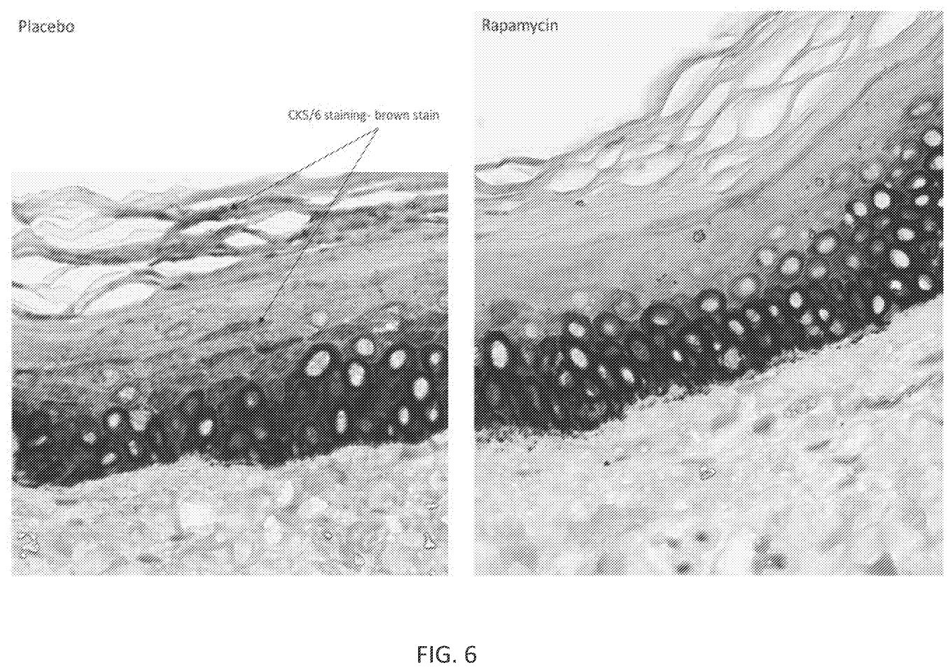

High magnification (40×) of CK 5/6 staining demonstrated, as shown in FIG. 6, a positive staining in the stratum granulosum and extending into the stratum corneum in the placebo treated skin, while the pattern of CK 5/6 staining remained restricted to the basal layer in the rapamycin treated biopsy.

### Example 7

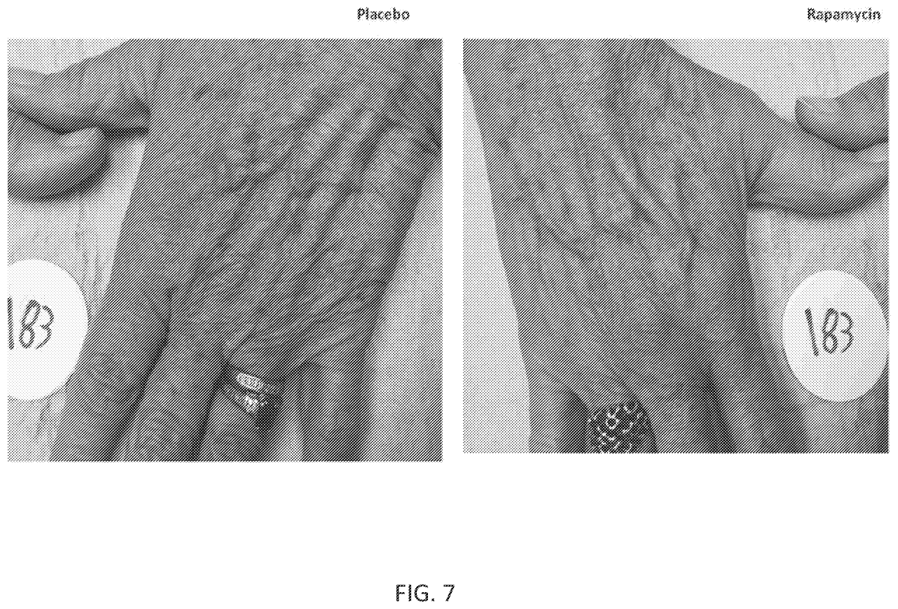

As shown in FIG. 7, clinical improvement in seen a 67 years old woman following the application of 10 μM (0.001%) rapamycin cream for 6 months. Placebo treated left hand is provided for comparison.

### Example 8

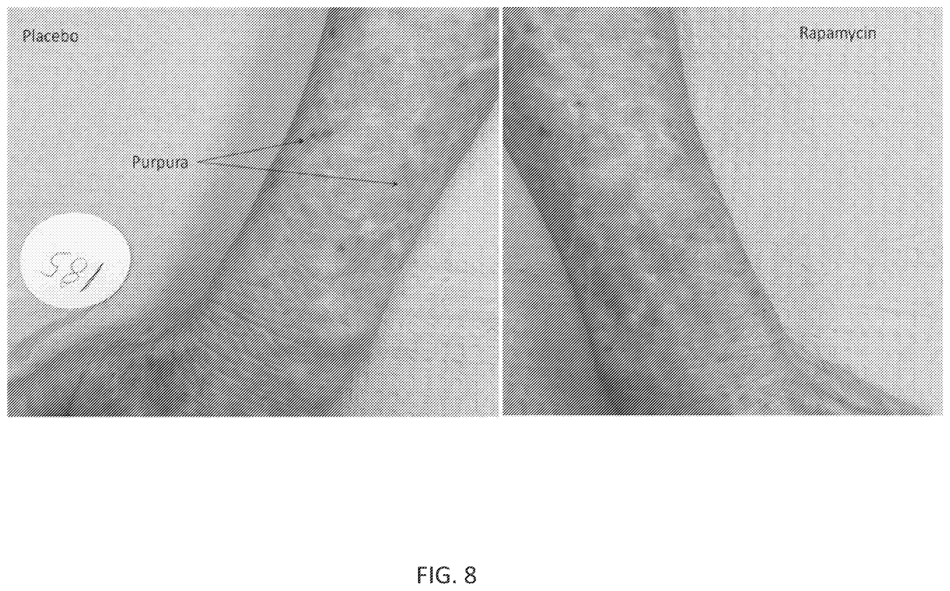

A Caucasian female, mid-sixties, was treated with rapamycin (0.001%) for 4.5 months. The patient was a cancer survivor, multiple round of chemotherapy, recurrent purpura with subsequent scarring. Results of the treatment are presented in in FIG. 8, which shows that there was a significant reduction in purpura following the 4.5 month treatment period. Treatment was discontinued at end of trial.

### Example 9

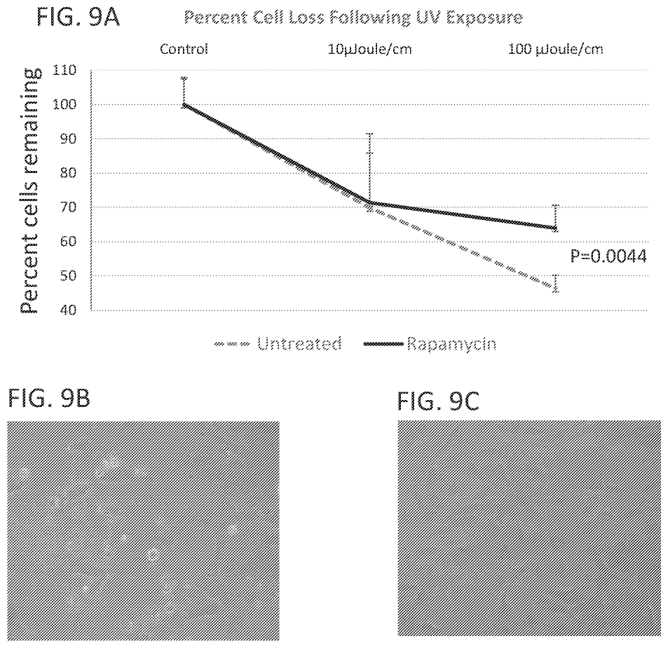

FIGS. 9A-9C show that rapamycin protects human cells from UV damage. Cells grown in the presence or absence of 1 nM rapamycin were exposed to increasing amounts of UV radiation and cell numbers were determined following 24 hours. The percent cell loss is presented in FIG. 9A. Rapamycin treated cultures showed a reduction in cell death following UV irradiation (P=0.0044). In FIG. 9B, a representative photograph of the control culture 24 hours following UV is presented. Note the typical hallmarks of apoptosis including rounded floating cells and typical cell blebbing. In FIG. 9C, a parallel culture which had been grown in 1 nM rapamycin is presented which has a notable lack of apoptotic cells.

### Enumerated Embodiments

The following exemplary embodiments are provided, the numbering of which is not to be construed as designating levels of importance.

Embodiment 1 provides a method of treating and/or preventing a dermal disorder in a mammalian subject in need thereof, the method comprises topically administering to the subject a composition comprising a therapeutically effective amount of rapamycin, or a salt, solvate, enantiomer or diastereoisomer thereof, wherein the dermal disorder is at least one selected form the group consisting of static wrinkles, fine wrinkles, loss of skin tone, ephelides, melasma, senile purpura, static rhytides, UV damage, prominent hand veins, field cancerization of epithelium such as in squamous cell carcinoma, keloids, and imbalance in ratio of collagen isoforms.

Embodiment 2 provides the method of Embodiment 1, wherein the field cancerization of epithelium comprises epidermal dysplasia and actinic damage.

Embodiment 3 provides the method of Embodiments 1-2, wherein the subject is human.

Embodiment 4 provides the method of Embodiments 1-3, wherein the composition comprises about 0.1 to about 0.0001% by weight of the rapamycin, or a salt, solvate, enantiomer or diastereoisomer thereof.

Embodiment 5 provides the method of Embodiments 1-4, wherein the composition comprises about 0.001% to about 0.0001% by weight of the rapamycin, or a salt, solvate, enantiomer or diastereoisomer thereof.

Embodiment 6 provides the method of Embodiments 1-5, wherein the composition further comprises a dermatologically acceptable carrier.

Embodiment 7 provides the method of Embodiments 1-6, wherein the dermatologically acceptable carrier is at least one selected from the group consisting of a solvent, lubricant, emollient, emulsifier, moisturizer, thickening wax, softener, fragrance, preservative, and artificial color.

Embodiment 8 provides the method of Embodiments 1-7, wherein the dermatologically acceptable carrier comprises petrolatum.

Embodiment 9 provides the method of Embodiments 1-8, wherein the composition is applied to the site of the dermal disorder of the subject.

Embodiment 10 provides a kit for treating and/or preventing a dermal disorder in a mammalian subject in need thereof, the kit comprising: a composition comprising a therapeutically effective amount of rapamycin or a salt, solvate, enantiomer or diastereoisomer thereof, an applicator, and instructions for topically administering the composition to the subject, wherein the dermal disorder is at least one selected form the group consisting of: static wrinkles, fine wrinkles, loss of skin tone, ephelides, melasma, senile purpura, static rhytides, prominent hand veins, field cancerization of epithelium, keloids, and imbalance in ratio of collagen isoforms.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or ameliorating a dermal disorder in a mammalian subject in need thereof, the method comprising topically administering to the subject a composition comprising a therapeutically effective amount of rapamycin, or a salt, solvate, enantiomer or diastereoisomer thereof,
wherein the dermal disorder is at least one selected from the group consisting of static wrinkles, fine wrinkles, loss of skin tone, ephelides, melasma, senile purpura, static rhytides, UV damage, prominent hand veins, field cancerization of epithelium, keloids, and imbalance in ratio of collagen isoforms.

2. The method of claim 1, wherein the field cancerization of epithelium comprises epidermal dysplasia and actinic damage.

3. The method of claim 1, wherein the subject is a human.

4. The method of claim 1, wherein the composition comprises about 0.1% to about 0.0001% by weight of the rapamycin.

5. The method of claim 1, wherein the composition comprises about 0.001% to about 0.0001% by weight of the rapamycin.

6. The method of claim 1, wherein the composition further comprises a dermatologically acceptable carrier.

7. The method of claim 6, wherein the dermatologically acceptable carrier is at least one selected from the group consisting of a solvent, lubricant, emollient, emulsifier, moisturizer, thickening wax, softener, fragrance, preservative, and artificial color.

8. The method of claim 6, wherein the dermatologically acceptable carrier comprises petrolatum.

9. The method of claim 1, wherein the composition is applied to the site of the dermal disorder of the subject.

10. A kit for treating or ameliorating a dermal disorder in a mammalian subject in need thereof, the kit comprising: a composition comprising a therapeutically effective amount of rapamycin or a salt, solvate, enantiomer or diastereoisomer thereof, an applicator, and instructions for topically administering the composition to the subject,
wherein the dermal disorder is at least one selected form the group consisting of: static wrinkles, fine wrinkles, loss of skin tone, ephelides, melasma, senile purpura, static rhytides, prominent hand veins, field cancerization of epithelium, keloids, and imbalance in ratio of collagen isoforms.

11. The method of claim 1, wherein the field cancerization of epithelium is derived from squamous cell carcinoma.

12. The kit of claim 10, wherein the field cancerization of epithelium is derived from squamous cell carcinoma.
